(19) United States
(12) Patent Application Publication
(10) Pub. No.: US 2013/0066032 A1
(43) Pub. Date: Mar. 14, 2013

(54) PROCESS FOR PREPARING ETHYLENE AND/OR PROPYLENE

(71) Applicants: Leslie Andrew CHEWTER, Amsterdam (NL); Rajaram RAMESH, Amsterdam (NL); Sivakumar SADASIVAN VIJAYAKUMARI, Amsterdam (NL); Jeroen VAN WESTRENEN, Amsterdam (NL)

(72) Inventors: Leslie Andrew CHEWTER, Amsterdam (NL); Rajaram RAMESH, Amsterdam (NL); Sivakumar SADASIVAN VIJAYAKUMARI, Amsterdam (NL); Jeroen VAN WESTRENEN, Amsterdam (NL)

(73) Assignee: SHELL OIL COMPANY, Houston, TX (US)

(21) Appl. No.: 13/606,239

(22) Filed: Sep. 7, 2012

(30) Foreign Application Priority Data
Sep. 7, 2011 (EP) 11180323.5

(51) Int. Cl. C07C 4/06; C08F 110/06; C08F 110/02; C07C 29/03; C07D 301/03

(52) U.S. Cl. 526/351; 585/300; 568/840; 549/523; 526/352

(57) ABSTRACT

The present invention provides a process for preparing ethylene and/or propylene, comprising providing a hydrocarbon stream, comprising C4+ normal and C4+ iso-olefins; subjecting the hydrocarbon stream to an etherification process wherein the iso-olefins are converted with alcohol to an tert-alkyl ether, and retrieving a first etherification product stream; separating the first etherification product stream into a first ether-enriched stream and an iso-olefin-depleted hydrocarbon stream; converting the normal olefins in the iso-olefin-depleted hydrocarbon stream to ethylene and/or propylene with a molecular sieve-comprising catalyst and retrieving an olefinic product; decomposing the tert-alkyl ether in the ether-enriched stream into alcohol and an iso-olefin; isomerising the obtained iso-olefins to normal-olefins and retrieving an normal-olefin-enriched hydrocarbon stream; and converting the normal olefins in the normal-olefin-enriched hydrocarbon stream to ethylene and/or propylene by contacting the normal-olefin-enriched hydrocarbon stream with a molecular sieve-comprising catalyst and retrieving an olefinic product.

This application claims the benefit of European Application No. 11180323.5 filed Sep. 7, 2011, which is incorporated herein by reference.

### FIELD OF THE INVENTION

The invention relates to a process for preparing ethylene and/or propylene.

### BACKGROUND TO THE INVENTION

Methanol-to-olefin processes are well described in the art. Typically, methanol-to-olefin processes are used to produce predominantly ethylene and propylene. An example of such a methanol-to-olefin process is described in WO-A 2006/020083. In the process of WO-A 2006/020083, the methanol is first converted into dimethylether (DME) prior to be subjected to a conversion to olefins, thereby reducing the amount of water produced during the conversion to olefins. Both methanol and DME are suitable feedstocks for a Methanol-to-olefin process and therefore such processes are also generally referred to as oxygenate-to-olefin (OTO) processes.

In U.S. Pat. No. 6,049,017, another OTO process is described wherein in addition to the conversion of oxygenates, also C4 olefins are converted to provide additional ethylene and propylene. In the process of U.S. Pat. No. 6,049,017, an oxygenate feedstock is converted to ethylene, propylene and additionally C4 olefins. The C4 olefins are separated from the ethylene and propylene and subsequently converted to ethylene and propylene in an olefin cracking process (OCP) over a SAPO catalyst. A disadvantage of the process described in U.S. Pat. No. 6,049,017 is that the SAPO-based OCP is less suitable for converting isobutene to ethylene and propylene, providing a conversion of no more of 15 wt % of the isobutene to ethylene and propylene. Therefore, in U.S. Pat. No. 6,049,017 the isobutene is separated from the remained of the C4 olefins by reacting the isobutene with methanol to MTBE. The MTBE is withdrawn from the process as a product. However, by doing so valuable feedstock, i.e. both methanol and C4 olefins, are removed from the process as an MTBE by-product rather than being converted to the desired ethylene and propylene.

There is a need in the art for an improved process for preparing ethylene and propylene wherein the production of by-product MTBE is reduced.

### SUMMARY OF THE INVENTION

It has now been found that it is possible to produce additional ethylene and propylene yield from the isobutene even is the catalyst is a SAPO-based catalyst such as SAPO-34, by converting the MTBE to ethylene and propylene.

Accordingly, the present invention provides a process for preparing ethylene and/or propylene, comprising the steps of:

a) providing a hydrocarbon stream, comprising C4+ normal olefins and C4+ iso-olefins;
b) subjecting the hydrocarbon stream to an etherification process with methanol and/or ethanol wherein at least part of the iso-olefins are converted with methanol and/or ethanol to an tert-alkyl ether, and retrieving a first etherification product stream;
c) separating at least part of the first etherification product stream into at least a first ether-enriched stream and an iso-olefin-depleted hydrocarbon stream;
d) converting at least part of the normal olefins in the iso-olefin-depleted hydrocarbon stream to ethylene and/or propylene by contacting at least part of iso-olefin-depleted hydrocarbon stream with a molecular sieve-comprising catalyst at a temperature in the range of from 200 to 1000° C. and retrieving an olefinic product comprising ethylene and/or propylene;
e) decomposing at least part of the tert-alkyl ether in the ether-enriched stream into methanol and/or ethanol and an iso-olefin by contacting the tert-alkyl-ether with an acid catalyst;
f) isomerising at least part of the obtained iso-olefins to normal-olefins in the presence of an isomerisation catalyst, and retrieving an normal-olefin-enriched hydrocarbon stream; and
g) converting at least part of the normal olefins in the normal-olefin-enriched hydrocarbon stream to ethylene and/or propylene by contacting at least part of the normal-olefin-enriched hydrocarbon stream with a molecular sieve-comprising catalyst at a temperature in the range of from 350 to 1000° C. and retrieving an olefinic product comprising ethylene and/or propylene.

By extracting iso-olefins from the hydrocarbon stream, comprising C4+ normal olefins and C4+ iso-olefins, in the form of a tert-alkyl ether and subsequently converting the tert-alkyl ether to further a normal butene enriched stream and subsequently ethylene and propylene in a next step, instead of withdrawing the MTBE as a by-product, the ethylene and propylene yield may be increased.

### BRIEF DESCRIPTION OF THE DRAWINGS

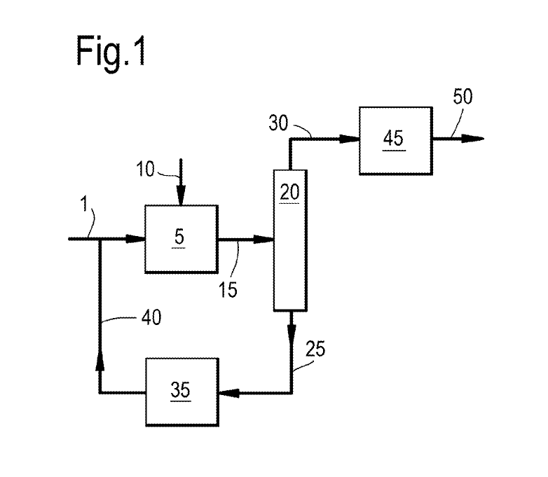

In FIG. 1, a schematic representation of a process according to the invention is provided.

### DETAILED DESCRIPTION OF THE INVENTION

In the process according to the present invention ethylene and/or propylene are prepared from a hydrocarbon stream, comprising C4+ normal olefins and C4+ iso-olefins. According to the invention the hydrocarbon stream is subjected to an etherification process with methanol and/or ethanol wherein at least part of the iso-olefins are converted with methanol and/or ethanol to a tert-alkyl ether. In the process according to the present invention at least part, and preferably all or essentially all, of the C4+ iso-olefins in the hydrocarbon stream are extracted from the hydrocarbon stream. The iso-olefins are extracted from the hydrocarbon stream by reacting iso-olefins with an alcohol, in particular methanol and/or ethanol to form tert-alkyl ethers, such as for example methyl tert-butyl ether (MTBE), ethyl tert-butyl ether (ETBE), tert-amyl methyl ether (TAME)or tert-amyl ethyl ether (TAEE). The formed ethers are separated from the remainder of the hydrocarbon stream. Only iso-olefins, wherein the double bound is located directly adjacent to a tertiary carbon atom, can react with methanol to form tert-alkyl ethers. Such iso-olefins are herein referred to as tertiary iso-olefins. Examples of such tertiary iso-olefins include isobutene, 2-methyl-1-butene and 2-methyl-2-butene. An example of an iso-olefin that is not a tertiary iso-olefin is 3-methyl-1-butene. Therefore, in the process according to the present invention at least part of the iso-olefins in the hydrocarbon stream should be tertiary iso-olefins.

At least part of the remainder of the hydrocarbon stream, which is now iso-olefin depleted, is converted to ethylene and/or propylene. This may for instance be done by for instance by providing the remainder of the hydrocarbon stream to an olefin cracking process (OCP) or by providing the remainder of the hydrocarbon stream to an oxygenate-to-olefins (OTO) process together with an oxygenate.

The tert-alkyl ethers, obtained by extracting the iso-olefins from the hydrocarbon stream by reacting the iso-olefins with methanol, are subsequently isomerised to normal olefins and subsequently converted to at least ethylene and propylene.

The process according to the invention is now described in more detail herein below.

In the process according to the invention, a hydrocarbon stream, comprising C4+ normal olefins, for example 1-butene, 2-butene, 1-pentene and/or 2-pentene, and C4+ iso-olefins, for example isobutene, 2-methyl-1-butene or 2-methyl-2-butene and optionally 3-methyl-1-butene, is provided.

Preferably, the hydrocarbon stream comprises at least C4 and/or C5 olefins, wherein the term olefins relates to both normal-olefins and iso-olefins, preferably at least C4 olefins. More preferably, the hydrocarbon stream comprises in the range of from 10 to 100 wt % of C4 and/or C5 olefins based on the weight of the olefins in the hydrocarbon stream, preferably of from 50 to 100 wt % of C4 and/or C5 olefins based on the weight of the olefins in the hydrocarbon stream. Even more preferably, the hydrocarbon stream comprises in the range of from 10 to 100 wt % of C4 olefins based on the weight of the olefins in the hydrocarbon stream, preferably of from 50 to 100 wt % of C4 olefins based on the weight of the olefins in the hydrocarbon stream. Optionally, the hydrocarbon stream also contains a diluent. Examples of suitable diluents include, but are not limited to, such as water or steam, nitrogen, argon, paraffins and methane.

Reference herein to hydrocarbons is to molecules comprising only carbon atoms and hydrogen atoms. Preferably, the hydrocarbon stream comprises in the range of from 10 to 100 wt % of olefins, wherein the term olefins relates to both normal-olefins and iso-olefins, based on the weight of the hydrocarbons in the hydrocarbon stream, preferably of from 60 to 100 wt % of C4 olefins based on the weight of the hydrocarbons in the hydrocarbon stream. Preferably, the hydrocarbon stream comprises in the range of from 1 to 60 wt % of iso-olefins based on the weight of the olefins in the hydrocarbon stream, preferably of from 10 to 50 wt % of iso-olefins based on the weight of the olefins in the hydrocarbon stream. Optionally, the hydrocarbon stream also contains a diluent. Examples of suitable diluents include, but are not limited to, such as water or steam, nitrogen, argon, C2-C3 paraffins and methane. One example of a suitable hydrocarbon stream is the C4 cut of a FCC effluent stream, which typically contains normal butenes, isobutylene and butanes. Of the C4 cut of an FCC effluent, 20 wt % is isobutylene and the ratio of C4 olefins to C4 paraffins is typically approximately 1.8. Another example of a suitable hydrocarbon stream is the C4 or C4/C5 cut of the product stream of an OTO process.

In step (b) of the process according to the invention the hydrocarbon stream is subjected to an etherification process. In the etherification process the hydrocarbon stream is contacted with an alcohol, preferably methanol and/or ethanol, in the presence of a suitable etherification catalyst. When the iso-olefins, preferably isobutylene, 2-methyl-1-butene or 2-methyl-2-butene in the hydrocarbon stream are contacted with the alcohol in the presence of an etherification catalyst, at least part of the iso-olefins are converted with the alcohol to tert-alkyl ethers. Reference herein in to a tert-alkyl ether is to an ether of an alcohol and an iso-olefin. Preferably, the alcohol is methanol and/or ethanol and the tert-alkyl ethers are methyl tert-butyl ether (MTBE), ethyl tert-butyl ether (ETBE), tert-amyl methyl ether (TAME) or tert-amyl ethyl ether (TAEE), which are tert-alkyl ethers of respectively methanol and ethanol with the mentioned iso-olefins. From the etherification process, a first etherification product stream is retrieved. The etherification product stream will comprise the formed tert-alkyl ethers and the remainder of the hydrocarbon stream, i.e. the unreacted components, including C4+ normal-olefins and optionally other hydrocarbons. In addition, the etherification product stream may also comprise unreacted alcohol. Typically the etherification reaction is performed in the presence of an excess of alcohol, i.e. above reaction stoichiometry with the iso-olefin.

At least part, and preferably all, of the first etherification product stream is separated in step (c) into at least an ether-enriched stream and an iso-olefin-depleted hydrocarbon stream, including C4+ normal olefins and optionally other hydrocarbons. The separation of the etherification product stream into an ether-enriched stream and an iso-olefin-depleted hydrocarbon stream can be done with normal separation means provided in the art. Due to the relatively high boiling points of methanol and ethanol, the bulk of the excess alcohol can be directed toward the ether-enriched stream.

In step (d) of the process according to the present invention, at least part of the normal olefins in the iso-olefin-depleted hydrocarbon stream is converted to ethylene and/or propylene by contacting at least part of iso-olefin-depleted hydrocarbon stream with a molecular sieve-comprising catalyst at a temperature in the range of from 350 to 1000° C. and retrieving an olefinic product comprising ethylene and/or propylene. Preferably, the olefinic product comprises advantageously at least 50 mol %, in particular at least 50 wt %, ethylene and propylene, based on total hydrocarbon content in the olefinic product. The olefinic product may also comprise C4+ olefins, which may be separated from the remainder of the olefinic product are for instance be recycled to the etherification process as part of the hydrocarbon stream. The molecular sieve-comprising catalyst may be a zeolite-comprising catalyst. However, the process according to the invention is particularly suitable to be used in case the molecular sieve-comprising catalyst in step (d) comprises a non-zeolitic molecular sieve, preferably a SAPO, AlPO or MeAlPO type molecular sieve, more preferably SAPO-34. Non-zeolitic molecular sieves are less suitable to convert iso-olefins, therefore process using these type of non-zeolitic molecular sieves benefit optimally from the fact that the iso-olefins are removed from the hydrocarbon stream prior to it being converted in step (d) to ethylene and propylene. Such a process wherein normal C4+ olefins are converted to an olefinic product comprising ethylene and/or propylene is also referred to as an olefin cracking process or OCP. Operating condition for such an OCP process are provided herein below

Preferably, in step (d) least part of the normal olefins in the iso-olefin-depleted hydrocarbon stream are contacted with the molecular sieve-catalyst together with an oxygenate, preferably at least one of methanol and dimethylether, more preferably methanol. When an oxygenate is provided together with the iso-olefin-depleted hydrocarbon stream, the process of step (d) is in fact an oxygenate-to-olefins (OTO) process and may be operated as such an OTO process. Operating condition for such an OTO process are provided herein below.

In step (e) of the process according to the invention, at least part of the tert-alkyl ether in the ether-enriched stream is converted decomposing the tert-alkyl ether in the ether-enriched stream into methanol and/or ethanol and an iso-olefin by contacting the tert-alkyl-ether with an acid catalyst;

In step (f) of the process according to the invention, at least part of the obtained iso-olefins are isomerised to normal-olefins in the presence of an isomerisation catalyst, and a normal-olefin-enriched hydrocarbon stream is retrieved; and

In step (g) of the process according to the invention, at least part of the normal olefins in the normal-olefin-enriched hydrocarbon stream is converted to ethylene and/or propylene by contacting at least part of the normal-olefin-enriched hydrocarbon stream with a molecular sieve-comprising catalyst at a temperature in the range of from 350 to 1000° C. and retrieving an olefinic product comprising ethylene and/or propylene. Preferably, the olefinic product comprises advantageously at least 50 mol %, in particular at least 50 wt %, ethylene and propylene, based on total hydrocarbon content in the olefinic product.

The tert-alkyl ethers in the ether-enriched steam will decompose back to methanol and/or ethanol (depending on the alcohol used in step (b)) and iso-olefin when contacted with an acid catalyst. This may be the same catalyst that was used in the etherification process, as the etherification reaction is an equilibrium reaction. Preferably, the temperature is increased above 150° C. when contacting the acid catalyst to favour the decomposing reaction. Following, the ether decomposition, the obtained iso-olefins subjected to a skeletal isomerisation, in the presence of an isomerisation catalyst, to isomerise at least part of the iso-olefins to normal-olefins.

Preferably, steps (e) and (f) are combined by contacting the ether-enriched stream directly with the isomerisation catalyst instead of first decomposing the tert-alkyl ether. Upon contacting the isomerisation catalyst, the tert-alkyl ether decomposes at least partly into its corresponding alcohol, i.e. methanol and/or ethanol, and iso-olefin, i.e. isobutene, in addition some minor amounts normal or secondary alkyl ethers may be formed. Isomerisation catalysts are acid catalyst and the decomposition reaction of the tert-alkyl ethers is also acid-catalysed. Therefore, the tert-alkyl ethers will at least partly decompose into the corresponding alcohol and iso-olefin as the ether-enriched stream is contacted with the isomerisation catalyst.

Alternatively, the ether-enriched stream is contacted with an acid catalyst, prior to contacting the isomerisation catalyst. This may for instance be done by passing oxygenate-comprising feedstock through an acid catalyst-comprising bed or by passing the feedstock through an acid grid or filter.

Following the decomposition of the ether into the corresponding alcohol and iso-olefin, at least part of the iso-olefins in the iso-olefin-depleted hydrocarbon stream undergo skeletal isomerisation to normal-olefins in the presence of the isomerisation catalyst.

The process of decomposing the tert-alkyl ether and subsequent isomerisation of the iso-olefin to normal olefins may be done using any isomerisation process suitable to induce skeletal isomerisation of iso-olefins to normal-olefins. Such process are well known in the art and commercially offered by several providers. Preferably, the iso-olefins are isomerized to normal-olefins by contacting the iso-olefins to with an isomerisation catalyst at a temperature in the range of from 200 to 350° C., preferably in the range of from 250 to 350° C. When the temperature is too low, no skeletal isomerisation will be achieved, while at higher temperatures oligomerisation and/or cracking of the olefins may occur.

Any isomerisation catalyst may be used that catalyses the skeletal isomerisation of normal-olefins to iso-olefins. Preferably, the isomerisation catalyst is a molecular sieve-comprising isomerisation catalyst. More preferably, an isomerisation catalyst comprising at least one of ferrierite, ZSM-22, ZSM-23, ZSM-35, SAPO-5, SAPO-11, SAPO-31, MeAPO-5, MeAPO-11, MeAPO-31, wherein Me is selected from the group of Mg, Mn, Co, Cr, and Fe. These catalysts combine low coke make with high selectivity.

Following decomposing the tert-alkyl ether and subsequent isomerisation of the iso-olefin to normal olefins, a normal-olefin-enriched hydrocarbon stream is retrieved from step (f), which can be converted under conditions similar to that of step (d). Preferably, at least part of the normal olefins in the normal-olefin-enriched hydrocarbon stream are converted by providing at least part of the normal olefins in the normal-olefin-enriched hydrocarbon stream to step (d). The later may be done by providing part or all of the normal-olefin-enriched hydrocarbon to step (d) together with the iso-olefin-depleted stream. However, preferably part or all of the normal-olefin-enriched hydrocarbon is provided together with the hydrocarbon stream to the etherification process in step (b). This is advantageous as the normal-olefin-enriched hydrocarbon may still contain residual iso-olefins, which can, at least partly, be captured and recycled to step (e) by separating them from the normal-olefins in steps (b) and (c).

In the process according to the invention, the hydrocarbon stream may be any hydrocarbon stream comprising C4+ normal olefins and C4+ iso-olefins. The hydrocarbon stream may be an external stream providing C4+ olefinic feedstock to the process, however it may also be an internal recycle stream, intended to recycle at least part of a C4+ hydrocarbon fraction from the effluent of an OTO reaction zone or OCP process to be used as a feed to the process in step (d). Examples of external hydrocarbon streams are the C4 and C5 fractions of the effluent of a refinery unit such as thermal cracking units, catalytic cracking units, steam cracking units, naphtha (steam) cracking units, butadiene extraction units and semi-hydrogenation units for removal of C4 and C5 diolefins. A particularly preferred C4 hydrocarbon stream is raffinate-1. Reference herein to raffinate-1 is to a stream comprising of isobutenes, normal butenes and mixed butanes and essentially no butadienes. Reference herein to essentially no butadienes is to a butadiene content of in the range of from 0 to 0.5 wt %, preferably 0 to 0.1, more preferably, 0 to 0.01 wt % of butadienes, based on the weight of the C4 hydrocarbons in the raffinate-1.

In the process according to the invention iso-olefins are reacted with methanol in an etherification process. The etherification process may be any suitable etherification process available in the art for etherifying methanol and iso-olefins to tert-alkyl ethers. Reference is made to the Handbook of MTBE and Other Gasoline Oxygenates, H. Hamid and M. A. Ali ed., 1^{st} edition, Marcel Dekker, New York, 2004, pages 65 to 223, where several established process and catalyst for preparing tert-alkyl ethers such as MTBE and ETBE are described. In particular reference is made to chapter 9, pages 203 to 220 of the Handbook of MTBE and Other Gasoline Oxygenates, wherein suitable commercial etherification processes are described. A preferred etherification process is an etherification process wherein the iso-olefins are converted with methanol to a tert-alkyl ether in the presence of a catalyst. Any homogeneous or heterogeneous Brönsted acid may be used to catalyze the etherification reaction. Such catalyst include: sulfuric acid, zeolites, pillared silicates, supported fluorocarbonsulphonic acid polymers and protonated cation-exchange resins catalyst, preferred catalyst are protonated cation-exchange resins catalyst due to the higher catalytic activity and the bound acid sites. A commonly used catalyst is Amberlyst 15.

Preferably, the iso-olefins are converted with an alcohol, preferably methanol and/or ethanol, more preferably methanol, to a tert-alkyl ether at a temperature in the range of from 30 to 100° C., more preferably 40 to 80° C. Preferably, the iso-olefins are converted with methanol and/or ethanol to a tert-alkyl ether at a pressures in the range of from 5 to 25 bar, more preferably 6 to 20 bar.

The iso-olefins may be converted with methanol and/or ethanol to a tert-alkyl ether in any etherification process, however, one preferred etherification process is based on a reactive distillation, which allows for a continuous etherification and separation of the formed ethers.

The hydrocarbon stream preferably contains little to no diolefins. Preferably, the hydrocarbon stream comprises in the range of from 0 to 0.5 wt %, preferably 0 to 0.1, more preferably, 0 to 0.01 wt % of butadienes, based on the weight of the hydrocarbons in the hydrocarbon stream. Most preferably, the hydrocarbon stream does not contain butadiene. Butadienes react to from undesired higher hydrocarbon compounds.

Preferably, the part of the hydrocarbon stream subjected to the etherification process is selectively hydrogenated to remove at least part of any diolefins, by hydrogenating the diolefins to mono-olefins and/or paraffins, preferably to mono-olefins.

Some methanol and/or ethanol may end up in streams other than the ether-enriched streams, such as the iso-olefins-depleted stream. This may be caused for example by the formation of azeotropic mixtures of methanol or ethanol with paraffinic or olefinic hydrocarbon components.

Methanol for instance may form an azeotropic mixture with normal butenes. The methanol concentration in this azeotropic mixture is approximately 4 wt %, based on weight of the azeotropic mixture. The ethanol may also form an azeotropic mixture with the butenes, wherein the ethanol concentration in the azeotropic mixture is approximately 2 wt %, based on weight of the azeotropic mixture. In the case of etherification with a mixed methanol/ethanol stream to produce for instance MTBE and ETBE, there are two different azeotropes. Methanol and ethanol may also form azeotropes with normal butanes and normal pentanes and normal pentenes. The methanol concentration in the azeotropic mixture with C5 normal olefins is approximately 12 wt %, based on weight of the azeotropic mixture. The ethanol concentration in the azeotropic mixture with C5 normal olefins is approximately 8 wt %, based on weight of the azeotropic mixture. The methanol concentration in the azeotropic mixture with normal pentane is approximately 9 wt %.

The presence of alcohol in the streams provided to the upstream processes will not negatively influence the desired reactions. However, it may be desired to remove the alcohol, prior to providing a stream to a further step of the process. Methanol and ethanol are a valuable feedstock for producing ethylene and propylene and is therefore preferably captured. Alcohol may be extracted from such streams by a water extraction. In one embodiment, alcohol is separated from hydrocarbons in an extraction column. Alcohols and hydrocarbons are fed to the bottom part of the extractor and water to the top section. The column is typically filled with random packing or sieve trays, which enhance alcohol mass-transfer from the hydrocarbon phase to the water phase. Essentially alcohol-free hydrocarbons may be retrieved above the water feed point, and a water/alcohol mixture is the bottom product. The alcohol may separated from the water by distillation and led back to the etherification process, or the water/alcohol mixture may be contacted with a molecular sieve to produce ethylene and/or propylene, for instance by providing the water/alcohol mixture to an OTO unit.

In the present invention, steps (d) and (g) may be operated as an oxygenate-to-olefins (OTO) process, by converting the normal olefins together with or as part of an oxygenate-comprising feedstock.

In such and OTO process, an oxygenate-comprising feedstock is contacted in an OTO zone with an oxygenate conversion catalyst under oxygenate conversion conditions, to obtain a conversion effluent comprising lower olefins. In the OTO zone, at least part of the feedstock is converted into an olefinic product, i.e. a product containing one or more olefins, including ethylene and/or propylene.

The oxygenate-comprising feedstock preferably comprises oxygenates, which comprise at least one oxygen-bonded alkyl group. The alkyl group preferably is a C1-C5 alkyl group, more preferably C1-C4 alkyl group, i.e. comprises 1 to 5, respectively, 4 carbon atoms; more preferably the alkyl group comprises 1 or 2 carbon atoms and most preferably one carbon atom. Examples of oxygenates that can be used in the oxygenate-comprising feedstock include alcohols and ethers. Examples of preferred oxygenates include alcohols, such as methanol, ethanol, propanol; and dialkyl ethers, such as dimethylether, diethylether, methylethylether. Preferably, the further oxygenate is methanol or dimethylether, or a mixture thereof.

Preferably the oxygenate-comprising feedstock comprises at least 50 wt % of oxygenate, based on total hydrocarbons and oxygenates in the oxygenate-comprising feedstock, more preferably at least 70 wt %.

The oxygenate feedstock can comprise an amount of diluents. During the conversion of the oxygenates, steam is produced as a by-product, which serves as an in-situ produced diluent. Optionally additional steam is added as diluent. The amount of additional diluent that needs to be added depends on the in-situ water make, which in turn depends on the composition of the oxygenate-comprising feed. Where the diluent is water or steam, the molar ratio of oxygenate to diluent is preferably between 10:1 and 1:20.

Preferably, in addition to the oxygenate, an olefinic co-feed is provided along with and/or as part of the oxygenate feedstock. Reference herein to an olefinic co-feed is to an olefin-comprising co-feed. In step (d) of the process the olefinic co-feed comprises or consists of the iso-olefin-depleted hydrocarbon stream and in step (g) the olefinic co-feed comprises or consists of the normal olefin-enriched stream. The olefinic co-feed preferably comprises C4 and higher olefins, more preferably C4 and C5 olefins. Preferably, the olefinic co-feed comprises at least 25 wt %, more preferably at least 50 wt %, of C4 olefins, and at least a total of 70 wt % of C4 hydrocarbon, based on weight of the olefinic co-feed.

Preferably, at least 70 wt % of the olefinic co-feed, during normal operation, is formed by a recycle stream of a C4+ hydrocarbon fraction from the OTO conversion effluent, preferably at least 90 wt % of olefinic co-feed, based on the whole olefinic co-feed, is formed by such recycle stream. In order to maximize production of ethylene and propylene, it is desirable to maximize the recycle of C4 olefins in the effluent of the OTO process. As described herein above, this can be done by recycling at least part of the C4+ hydrocarbon fraction, preferably a C4-C5 hydrocarbon fraction, more preferably C4 hydrocarbon fraction, in the olefinic product, which is retrieved as the OTO effluent. However, a certain part thereof, such as between 1 and 5 wt %, needs to be withdrawn as purge, since otherwise saturated hydrocarbons, in particular C4 saturated hydrocarbons (butane) would build up in the process, which are substantially not converted under the OTO reaction conditions. Preferably, the saturated hydrocarbons are withdrawn from the process using a the process according to the present invention wherein at least part of the C4+ hydrocarbon fraction retrieved as the OTO effluent, forms at least part of the hydrocarbon stream provided in step (a).

The preferred molar ratio of oxygenate in the oxygenate feedstock to olefin in the olefinic co-feed provided to the OTO conversion zone depends on the specific oxygenate used and the number of reactive oxygen-bonded alkyl groups therein. Preferably the molar ratio of oxygenate to olefin in the total feed, i.e. oxygenate feed and olefinic co-feed, lies in the range of 20:1 to 1:10, more preferably in the range of 18:1 to 1:5, still more preferably in the range of 15:1 to 1:3, even still more preferably in the range of 12:1 to 1:3.

A variety of OTO processes is known for converting oxygenates to an olefin-containing product, as already referred to above. One such process is described in WO-A 2006/020083. Processes integrating the production of oxygenates from synthesis gas and their conversion to light olefins are described in US20070203380A1 and US20070155999A1.

Catalysts suitable for converting normal-olefins in the iso-olefin-depleted stream in step (d), optionally together with any oxygenates provided to step (d), to an olefinic product comprising at least ethylene and/or propylene include molecular sieve-comprising catalyst compositions.

The molecular sieve-comprising catalyst compositions typically also include binder materials, matrix material and optionally fillers. Suitable matrix materials include clays, such as kaolin. Suitable binder materials include silica, alumina, silica-alumina, titania and zirconia, wherein silica is preferred due to its low acidity.

Molecular sieves preferably have a molecular framework of one, preferably two or more corner-sharing [TO₄] tetrahedral units, more preferably, two or more [SiO₄], [AlO₄] and/or [PO₄] tetrahedral units. These silicon, aluminum and/or phosphorous based molecular sieves and metal containing silicon, aluminum and/or phosphorous based molecular sieves have been described in detail in numerous publications including for example, U.S. Pat. No. 4,567,029. In a preferred embodiment, the molecular sieves have 8-, 10- or 12-ring structures and an average pore size in the range of from about 3 Å to 15 Å.

Suitable molecular sieves are silicoaluminophosphates (SAPO), such as SAPO-17, -18, -34, -35, -44, but also SAPO-5, -8, -11, -20, -31, -36, -37, -40, -41, -42, -47 and -56; aluminophosphates (AlPO) and metal substituted (silico)aluminophosphates (MeAlPO), wherein the Me in MeAlPO refers to a substituted metal atom, including metal selected from one of Group IA, IIA, IB, IIIB, IVB, VB, VIB, VIIB, VIIIB and Lanthanide's of the Periodic Table of Elements, preferably Me is selected from one of the group consisting of Co, Cr, Cu,Fe, Ga, Ge, Mg, Mn, Ni, Sn, Ti, Zn and Zr.

Alternatively, the conversion of the normal olefins in the iso-olefins-depleted stream and optionally the conversion of oxygenates may be accomplished by the use of an aluminosilicate-comprising catalyst, in particular a zeolite-comprising catalyst. Suitable catalysts include those containing a zeolite of the ZSM group, in particular of the MFI type, such as ZSM-5, the MTT type, such as ZSM-23, the TON type, such as ZSM-22, the MEL type, such as ZSM-11, the FER type. Other suitable zeolites are for example zeolites of the STF-type, such as SSZ-35, the SFF type, such as SSZ-44 and the EU-2 type, such as ZSM-48.

Aluminosilicate-comprising catalysts, and in particular zeolite-comprising catalysts, have the additional advantage that in addition to the conversion of methanol or ethanol, these catalysts also induce the conversion of iso-olefins to ethylene and/or propylene.

In one preferred embodiment, the molecular sieve in the molecular sieve-comprising catalyst of step (d) is a non-zeolitic molecular sieve.

The normal olefins in the iso-olefin depleted stream, and optionally any oxygenates, may be converted in step (d) using a catalyst comprising a more-dimensional zeolite, in particular of the MFI type, more in particular ZSM-5, or of the MEL type, such as zeolite ZSM-11. Such zeolites are particularly suitable for converting olefins, including iso-olefins, to ethylene and/or propylene. The zeolite having more-dimensional channels has intersecting channels in at least two directions. So, for example, the channel structure is formed of substantially parallel channels in a first direction, and substantially parallel channels in a second direction, wherein channels in the first and second directions intersect. Intersections with a further channel type are also possible. Preferably the channels in at least one of the directions are 10-membered ring channels. A preferred MFI-type zeolite has a Silica-to-Alumina ratio SAR of at least 60, preferably at least 80. The catalyst can comprise at least 1 wt %, based on total molecular sieve in the catalyst, of the molecular sieve having more-dimensional channels, preferably at least 5 wt %, more preferably at least 8 wt %.

One particular group of catalysts includes catalysts comprising one or more zeolite having one-dimensional 10-membered ring channels, i.e. one-dimensional 10-membered ring channels, which are not intersected by other channels. Preferred examples are zeolites of the MTT and/or TON type. Preferably, the catalyst comprises at least 40 wt %, preferably at least 50% wt of such zeolites based on total zeolites in the catalyst. Such catalyst may comprise in addition to one or more one-dimensional zeolites having 10-membered ring channels, such as of the MTT and/or TON type, a more-dimensional zeolite, in particular of the MFI type, more in particular ZSM-5, or of the MEL type, such as zeolite ZSM-11.

The molecular sieve catalyst may comprise phosphorous as such or in a compound, i.e. phosphorous other than any phosphorous included in the framework of the molecular sieve. It is preferred that an MEL or MFI-type zeolites comprising catalyst additionally comprises phosphorous. The phosphorous may be introduced by pre-treating the MEL or MFI-type zeolites prior to formulating the catalyst and/or by post-treating the formulated catalyst comprising the MEL or MFI-type zeolites. Preferably, the catalyst comprising MEL or MFI-type zeolites comprises phosphorous as such or in a compound in an elemental amount of from 0.05-10 wt % based on the weight of the formulated catalyst. A particularly preferred catalyst comprises phosphor-treated MEL or MFI-type zeolites having SAR of in the range of from 60 to 150, more preferably of from 80 to 100. An even more particularly preferred catalyst comprises phosphor-treated ZSM-5 having SAR of in the range of from 60 to 150, more preferably of from 80 to 100.

It is preferred that molecular sieves in the hydrogen form are used in the oxygenate conversion catalyst in step (g), e.g., HZSM-22, HZSM-23, and HZSM-48, HZSM-5. Preferably at least 50% w/w, more preferably at least 90% w/w, still more preferably at least 95% w/w and most preferably 100% of the total amount of molecular sieve used is in the hydrogen form. It is well known in the art how to produce such molecular sieves in the hydrogen form.

The reaction conditions for an OCP process step, i.e. the conversion of olefins in the absence of oxygenates, and an OTO process step, i.e. the conversion of oxygenates, optionally together with olefins, include a reaction temperature of 350 to 1000° C., preferably from 350 to 750° C., more preferably 450 to 700° C., even more preferably 500 to 650° C.; and a pressure from 0.1 kPa (1 mbar) to 5 MPa (50 bar), preferably from 100 kPa (1 bar) to 1.5 MPa (15 bar).

As mention herein above, in case of an OTO processes step, preferably a diluent is present. The same applies to an OCP process step. The hydrocarbon stream and the resulting iso-olefin-depleted stream can comprise an amount of diluents. One suitable diluent is water. Other suitable diluents include inert gases such as nitrogen, but may also include paraffins. Where the diluent is water or steam, the molar ratio of olefins to diluent is between 10:1 and 1:20.

Typically the catalyst deactivates in the course of the process, primarily due to deposition of coke on the catalyst. Conventional catalyst regeneration techniques can be employed to remove the coke. It is not necessary to remove all the coke from the catalyst as it is believed that a small amount of residual coke may enhance the catalyst performance and additionally, it is believed that complete removal of the coke may also lead to degradation of the molecular sieve.

The catalyst particles used in the process of the present invention can have any shape known to the skilled person to be suitable for this purpose, for it can be present in the form of spray dried catalyst particles, spheres, tablets, rings, extrudates, etc. Extruded catalysts can be applied in various shapes, such as, cylinders and trilobes. If desired, spent oxygenate conversion catalyst can be regenerated and recycled to the process of the invention. Spray-dried particles allowing use in a fluidized bed or riser reactor system are preferred. Spherical particles are normally obtained by spray drying. Preferably the average particle size is in the range of 1-200 μm, preferably 50-100 μm.

Step (d) of the process may be operated in a fluidized bed or moving bed, e.g. a fast fluidized bed or a riser reactor system, and also in a fixed bed reactor or a tubular reactor. A fluidized bed or moving bed, e.g. a fast fluidized bed or a riser reactor system are preferred. The same applies when converting the tert-alkyl ethers in the embodiment, wherein in step (e) the tert-alkyl ethers in the ether-enriched stream are converted to an olefinic product comprising ethylene and/or propylene. In step (d) and (g) of the process an olefinic product stream comprising ethylene and/or propylene is retrieved. The ethylene and/or propylene may be separated from the remainder of the components in the olefinic products. Preferably the olefinic product and further olefinic product at least partially, and preferably fully, combined prior to separating the ethylene and/or propylene from the remaining components. Where the olefinic product comprises ethylene, least part of the ethylene may be further converted into at least one of polyethylene, mono-ethylene-glycol, ethylbenzene and styrene monomer. Where the olefinic product comprises propylene, at least part of the propylene may be further converted into at least one of polypropylene and propylene oxide.

### DETAILED DESCRIPTION OF THE FIGURES

In FIG. 1, a process according to the present invention is schematically represented. In FIG. 1, hydrocarbon stream 1, comprising C4+ normal olefins and C4+ iso-olefins is provided to etherification zone 5, together with methanol 10. In etherification zone 5, hydrocarbon stream 1 is contacted with methanol 10 over an etherification catalyst, such as for instance a protonated cationic-exchange resin. Etherification product stream 15 is retrieved from etherification zone 5 and provided to separation zone 20, wherein etherification product 15 is separated into ether-enriched stream 25 and iso-olefin-depleted hydrocarbon stream 30. Optionally, zones 5 and 20 are combined into a reactive distillation zone, wherein iso-olefins are reacted with methanol to tert-alkyl ethers, while continuously separating tert-alkyl ether from the reaction mixture. Optionally, zones 5 and 20 allow for the recycle of part of the iso-olefin depleted stream in case not all of the iso-olefins are converted to tert-alkyl ether in a single pass process.

Ether-enriched streams 25 is retrieved from separation zone 20 and provided to skeletal isomerisation zone 35. In skeletal isomerisation zone 35, at least part of the tert-alkyl ethers are decomposed to the corresponding alcohol and iso-olefins in the presence of an isomerisation catalyst, such as for example SAPO-11 or Ferrierite. The iso-olefins are subsequently subjected to a skeletal isomerisation to normal-olefins in the presence of the same isomerisation catalyst. Normal-olefin-enriched hydrocarbon stream 40 is retrieved from skeletal isomerisation zone 35 and provided back to etherification zone 5.

Iso-olefin depleted hydrocarbon stream 30 is provided to olefin cracking zone 45. In olefin cracking zone 45, iso-olefin depleted hydrocarbon stream 30 is contacted with a molecular sieve-comprising catalyst, for example a catalyst comprising SAPO-34 or ZSM-5. Olefinic product 50, comprising ethylene and/or propylene is retrieved from olefin cracking zone 45. Optionally, a C4+ fraction comprised in olefinic product 50 is recycled to etherification unit 1 to further enhance ethylene and propylene yield (not shown). Optionally, olefin cracking zone 45 is replaced by a oxygenate-to-olefin zone and additionally one or more oxygenates, such as methanol or dimethylether, olefins and water are added to oxygenate-to-olefin zone (not shown).

What is claimed is:

1. A process for preparing ethylene and/or propylene, comprising the steps of:
a) providing a hydrocarbon stream, comprising C4+ normal olefins and C4+ iso-olefins;
b) subjecting the hydrocarbon stream to an etherification process with methanol and/or ethanol wherein at least part of the iso-olefins are converted with methanol and/or ethanol to an tert-alkyl ether, and retrieving a first etherification product stream;
c) separating at least part of the first etherification product stream into at least a first ether-enriched stream and an iso-olefin-depleted hydrocarbon stream;
d) converting at least part of the normal olefins in the iso-olefin-depleted hydrocarbon stream to ethylene and/or propylene by contacting at least part of iso-olefin-depleted hydrocarbon stream with a molecular sieve-comprising catalyst at a temperature in the range of from 200 to 1000° C. and retrieving an olefinic product comprising ethylene and/or propylene;
e) decomposing at least part of the tert-alkyl ether in the ether-enriched stream into methanol and/or ethanol and an iso-olefin by contacting the tert-alkyl-ether with an acid catalyst;
f) isomerising at least part of the obtained iso-olefins to normal-olefins in the presence of an isomerisation catalyst, and retrieving an normal-olefin-enriched hydrocarbon stream; and
g) converting at least part of the normal olefins in the normal-olefin-enriched hydrocarbon stream to ethylene and/or propylene by contacting at least part of the normal-olefin-enriched hydrocarbon stream with a molecular sieve-comprising catalyst at a temperature in the range of from 350 to 1000° C. and retrieving an olefinic product comprising ethylene and/or propylene.

2. A process according to claim 1, wherein steps (e) and (f) are combined by contacting the ether-enriched stream with the isomerisation catalyst of step (f)

3. A process according to claim 1, wherein the iso-olefins and/or the ether-enriched stream is contacted with the isomerisation catalyst at a temperature in the range of from 200 to 350° C.

4. A process according to claim 1, wherein the at least part of the normal olefins in the normal-olefin-enriched hydrocarbon stream obtained in step (f) are converted by providing at least part of the normal olefins in the normal-olefin-enriched hydrocarbon stream to step (d) of the process.

5. A process according to claim 1, wherein the molecular sieve-comprising catalyst comprises a non-zeolitic molecular sieve, preferably least one of a SAPO, AlPO, or MeAlPO type molecular sieve.

6. A process according to claim 1, wherein in step (d) least part of the normal olefins in the iso-olefin-depleted hydrocarbon stream are contacted with the molecular sieve-catalyst together with an oxygenate.

7. A process according to claim 1, wherein the iso-olefins are converted with methanol and/or ethanol to the tert-alkyl ether by contacting the iso-olefin with methanol and/or ethanol in the presence of an etherification catalyst at a temperature in the range of from 30 to 100° C.

8. A process according to claim 7, wherein the etherification catalyst is a protonated cation-exchange resin catalyst.

9. A process according to claim 1, wherein in step (b) the iso-olefins are converted with methanol to a tert-alkyl ether.

10. A process according to claim 1, wherein the hydrocarbon stream comprises less than 0.5 wt % of diolefins, based on the weight of the hydrocarbons in the hydrocarbon stream.

11. A process according to claim 1, wherein the olefinic product comprises ethylene and at least part of the ethylene is further converted into at least one of polyethylene, mono-ethylene-glycol, ethylbenzene and styrene monomer.

12. A process according to claim 1, wherein the olefinic product comprises propylene and at least part of the propylene is further converted into at least one of polypropylene and propylene oxide.
